(19) United States
(12) Patent Application Publication
(10) Pub. No.: US 2022/0362273 A1
(43) Pub. Date: Nov. 17, 2022

(54) COMBINATION THERAPY FOR TREATING OR PREVENTING SKIN DAMAGE

(71) Applicants: Northwestern University, Evanston, IL (US); Case Western Reserve University, Cleveland, OH (US)

(72) Inventor: Kurt Q. Lu, Cleveland, OH (US)

(21) Appl. No.: 17/302,819

(22) Filed: May 12, 2021

(51) Int. Cl. A61K 31/7016; A61K 31/593; A61K 31/436; A61K 31/585; A61K 31/341; A61K 31/549; A61K 31/522; A61K 36/28; A61K 9/00; A61P 17/02

(52) U.S. Cl. CPC A61K 31/7016; A61K 31/593; A61K 31/436; A61K 31/585; A61K 31/341; A61K 31/549; A61K 31/522; A61K 36/28; A61K 9/0053; A61P 17/02

(57) ABSTRACT

In an aspect of the present application, a composition for preventing or treating skin damage in skin of a mammalian subject in need thereof can include at least one autophagy promoter and at least one diuretic. The at least one autophagy promoter and the at least one diuretic, in combination, can each be present in an amount sufficient to prevent or treat the skin damage. In another aspect, the present application includes a method for preventing or treating skin damage in skin of a mammalian subject in need thereof whereby the composition can be administered to the subject in a therapeutically effective amount to prevent or treat the skin damage.

### TECHNICAL FIELD

The present application relates generally to compositions and methods useful for treating or preventing skin damage and, in particular, to compositions comprising at least one autophagy promoter in combination with at least one diuretic useful in the prevention and treatment of skin damage in a mammalian subject in need thereof.

### BACKGROUND

Skin exposure to vesicants, such as nitrogen mustard and sulfur mustard causes direct damage to the skin, which initiates a physiologic response by the innate immune system. Specifically, inflammatory macrophages mobilize to the site of exposure where their activities further exacerbate local skin destruction. However, the historic use of mustards as weaponized agents from World War I to the modern era has demonstrated that the detrimental consequences of mustard extend beyond exposed surfaces of the skin, eyes, and lungs to include severe bone marrow suppression and death. Further, the skin lesion still suffers from a surge of cytokine storm that increases erythema and skin swelling accentuating pain.

### SUMMARY

The present application relates generally to compositions and methods useful for treating or preventing skin damage and, in particular, to compositions comprising at least one autophagy promoter in combination with at least one diuretic useful in the prevention and treatment of skin damage in a mammalian subject in need thereof.

Tissue injury from burns or exposure to toxicants initiates a series of reactions that call into action release of inflammatory mediators at the site of injury. In response, vascular leakage prompts influx of inflammatory cells including macrophages to the site of injury evolving into a microenvironment with increased osmolarity and high Na+ concentration. While increased Na+ concentration supports inflammation and cellular activation with upregulation of anti-microbial defenses in skin, an increase in osmolarity in the injured tissue microenvironment prevents the natural turnover of infiltrating macrophages by doubling their half-life through down-regulation of anti-apoptotic factors and augmenting macrophage polarization to the activated M1 inflammatory phenotype. Advantageously, and without being bound by theory, it is believed that the compositions and methods of the present application synergistically mitigate early inflammatory influx of macrophages in response to chemotactic mediators at the site of injury while also reducing swelling and pain at the site of injury to improve skin wound healing.

In an aspect of the present application, a composition for preventing or treating skin damage in skin of a mammalian subject in need thereof can comprise at least one autophagy promoter and at least one diuretic. The at least one autophagy promoter and the at least one diuretic, in combination, can each be present in an amount sufficient to prevent or treat the skin damage.

In another aspect of the present application, a method for preventing or treating skin damage in skin of a mammalian subject in need thereof can comprise administering to the subject a therapeutically effective amount of a composition comprise at least one autophagy promoter and at least one diuretic, thereby treating the skin damage in the subject. The at least one autophagy promoter and the at least one diuretic, in combination, can each be present in an amount sufficient to prevent or treat the skin damage.

In another aspect of the present application, a method for preventing or treating skin damage in skin of a mammalian subject in need thereof can comprise (i) administering, to the subject, a therapeutically effective amount of a composition comprising at least one autophagy promoter and at least one diuretic; and (ii) administering, after step (i), a therapeutically effect amount of a series of diuretic compositions on separate consecutive days to the subject; whereby steps (i) and (ii) are effective to prevent or treat the skin damage in the subject.

In another aspect of the present application, a kit is provided for preventing or treating skin damage in skin of a mammalian subject in need thereof. The kit can comprise a therapeutically effective amount of a composition comprising at least one autophagy promoter and at least one diuretic. The composition can be formulated for oral administration. The kit can further comprise a series of diuretic compositions, each of which is formulated for oral administration. Additionally, the kit can comprise a set of dosing instructions, wherein the subject is instructed to (i) ingest the composition on a first day, and (ii) subsequently ingest each of the diuretic compositions on separate consecutive days.

### BRIEF DESCRIPTION OF THE DRAWINGS

The foregoing and other features of the present application will become apparent to those skilled in the art to which the present application relates upon reading the following description with reference to the accompanying drawings, in which:

FIGS. 1A-C show the results of intervention with spironolactone (SP) monotherapy and combination treatment with Vitamin D. (FIG. 1A) Daily wound progression after exposure to topical nitrogen mustard (NM) (26.6 mg/kg). (FIG. 1B) Histological data of epidermal and dermal thickness of NM-challenged skin following treatment with 20 mg/kg SP alone or in combination with 5 ng 25OHD. (FIG. 1C) Relative iNOS gene expression 48 hours post NM challenge. Mice were treated with SP at 20 mg/kg or SP+5 ng 25 OHD (n=3 for all groups);

FIG. 2 is a graph showing skin thickness by bi-fold caliper measurements after NM exposure. Control group=0.05% topical NM exposure. Spironolactone group=NM exposure with i.p. spironolactone 20 mg/kg. Vitamin D was given on day 0. Spironolactone was given daily for 4 days;

FIG. 3 is a longitudinal graph of bi-fold thickness of mice exposed to 0.5% NM. Intervention treatment with spironolactone (200 mg/kg) was by gavage given on days 0, 1 and 2. Vitamin D (i.p. 5 ng in mineral oil) was given on day 0, 2 hours after NM exposure. Spironolactone+vitamin D group in drugs doses similar to single drug conditions. All animals including controls were subjected to similar sham oral oral gavage or mineral vehicle treatment;

FIG. 4 is a series of gross images of skin after NM exposure by daily photographs. Area of NM exposure is a 12 mm circle on the dorsal back of C57Bl/6 mice as marked by black marker. NM control=40 μl of 0.05% exposure. SP=oral gavage of spironolactone given on days 0, 1, 2. VD=5 ng i.p. vitamin D3 (25OHD3). VD+SP=combination treatment. The area of scab formation was analyzed by image-J™ software and quantified for all groups;

FIG. 5 is a series of graphs showing quantification of area of scab formation. Data was obtained using measurements from image-J™ software Area using formula (area of red scab/area of 12 mm NM exposure). NM control=40 μl of 0.05% exposure. SP=oral gavage of spironolactone given on days 0, 1, 2. VD=5 ng i.p. vitamin D3 (25OHD3). VD+SP=combination treatment (n=7-9 for different groups, p-value indicated in chart by ANOVA);

FIG. 6 is a series of graphs showing longitudinal quantification of area of scab formation until healing. Data was obtained using measurements from image-J™ software Area using formula (area of red scab/area of 12 mm NM exposure). NM control=40 μl of 0.05% exposure. SP=oral gavage of spironolactone given on days 0, 1, 2. VD=5 ng i.p. vitamin D3 (25OHD3). VD+SP=combination treatment (n=7-9 for different groups, p-value indicated in chart by ANOVA);

FIG. 7 is a series of graphs showing the results of qRT-PCR of skin exposed to NM at day 5. Significant reductions were observed for these five inflammatory markers with drug intervention. NM control=40 μl of 0.05% exposure. SP=oral gavage of spironolactone given on days 0, 1, 2. VD=5 ng i.p. vitamin D3 (25OHD3). VD+SP=combination treatment. P-values indicated on graphs; and

FIG. 8 is a schematic illustration showing one example of a kit for preventing or treating skin damage in skin of a mammalian subject in need thereof constructed in accordance with one aspect of the present application.

### DETAILED DESCRIPTION

### Definitions

All scientific and technical terms used in the present application have meanings commonly used in the art unless otherwise specified. The definitions provided herein are to facilitate understanding of certain terms used frequently herein and are not meant to limit the scope of the present application.

In the context of the present application, the term “A” or “an” means herein one or more than one; at least one. Where the plural form is used herein, it generally includes the singular.

“Comprising” means, without other limitation, including the referent, necessarily, without any qualification or exclusion on what else may be included. For example, “a composition comprising x and y” encompasses any composition that contains x and y, no matter what other components may be present in the composition. Likewise, “a method comprising the step of x” encompasses any method in which x is carried out, whether x is the only step in the method or it is only one of the steps, no matter how many other steps there may be and no matter how simple or complex x is in comparison to them. “Comprised of and similar phrases using words of the root “comprise” are used herein as synonyms of “comprising” and have the same meaning.

“Comprised of” is a synonym of “comprising” (see above).

Use of the term “includes” is not intended to be limiting.

“Effective route” can refer to a route that provides for delivery of a composition of the present application to a desired compartment, system, or location. For example, an effective route is one through which a composition of the present application can be administered to provide at a desired site of action (e.g., a site of skin damage) an amount of the composition sufficient to effectuate a beneficial or desired clinical result (e.g., preventing or treating skin damage).

The term “skin damage” can refer to any type of skin damage that is caused by one or more of chemical burns caused, for example, by vesicants (e.g., weaponized vesicants, such as mustard gas, nitrogen mustard and sulfur mustard, and chemotherapeutic vesicants, such as mechlorethamine and doxorubicin), a radiation burn (e.g., radiotherapy/radiation therapy as part of a cancer treatment, or UV- or X-ray induced skin damage), a heat burn (e.g., caused by fire, steam, hot objects or hot liquids), a cold burn, an electrical burn, a friction burn, trauma, surgical and post-surgical wounds and wound healing, and other types of breakdown of the stratum corneum, epidermis, and underlying tissues. UV-induced skin damage, for example, can refer to skin damage resulting from exposure to ultraviolet light in the A (320-400 nm), B (290-320 nm), or C ranges (200-290 nm). Skin damage can include, by way of example, erythema, edema, hyperpigmentation, dry desquamation, moist desquamation, epilation and ulceration.

“Subject” can mean a vertebrate, such as a mammal (e.g., a human). Mammals include, but are not limited to, humans, dogs, cats, horses, cows, and pigs.

“Autophagy” can refer to a variety of tightly-regulated catabolic processes that involve the degradation of a cell's own components through the lysosomal machinery and play a normal part in cell growth, development, and homeostasis, helping to maintain a balance between the synthesis, degradation, and subsequent recycling of cellular products. The most well-known catabolic process of autophagy involves the formation of a membrane around a targeted region of the cell, separating the contents from the rest of the cytoplasm. The resultant vesicle then fuses with a lysosome and subsequently degrades the contents.

The term “autophagy promoter” can refer to any agent, compound, or moiety capable of promoting and/or inducing autophagy in a cell. Whether or not an agent, compound, or moiety is an autophagy promoter, or has autophagy-promoting effects (e.g., in vitro or in vivo), can be assessed, for example, by evaluating the capacity or efficacy of the agent, compound, or moiety for clearance in cells; in other words, the autophagy activity of the agent, compound, or moiety. See U.S. Patent Publication No. 2012/01788119 for examples of methods to assess autophagy activity. When autophagy activity is higher, the clearance is regarded as functioning in living cells. When autophagy normally takes place, cellular homeostasis is considered to be maintained. Non-limiting examples of autophagy promoters that can be used as part of the present application are described below.

The term “diuretic” can refer to any agent, compound, or moiety that promotes (1) the excretion of urine (and, thus, a reduction in blood plasma volume) and/or (2) the reduction of swelling or edema. Non-limiting examples of diuretics include those compounds disclosed in reference texts, such as Martindale 32^{nd} Edition The Complete Drug Reference, especially page 778. Diuretics that may be used as part of the present application include any of those (diuretics) which are commonly divided into classes according to their mode of action, such as: carbonic anhydrase inhibitors; loop diuretics; potassium-sparing diuretics; and thiazides. Non-limiting examples of diuretics that can be used as part of the present application are described below. Also included in the term are diuretic-like compounds, e.g., compounds that have diuretic activity but may not be conventionally defined as diuretics.

The terms “prevent” or “preventing” can mean complete prevention, or a delay in the appearance, of a symptom or effect of skin damage. The terms can also include prophylactic uses and applications of the compositions of the present application.

The term “therapeutically effective amount” can refer to the amount of a composition of the present application determined to produce any therapeutic response in a subject. For example, effective combination therapy (i.e., compositions of the present application) may prolong the survivability of the subject and/or inhibit overt clinical symptoms. Treatments that are therapeutically effective within the meaning of the term as used herein include treatments that minimize injury at a site of skin damage and/or promote healing at a site of skin damage and/or reduce or prevent swelling at a site of skin damage and/or improve a subject's quality of life. Such therapeutically effective amounts are readily ascertained by one of ordinary skill in the art. Thus, to “treat” means to deliver such an amount.

“Treat,” “treating,” or “treatment” are used broadly in relation to the present application and each such term encompasses, among others, ameliorating, inhibiting, or curing a deficiency, dysfunction, disease, or other deleterious process, including those that interfere with and/or result from a therapy.

“Pharmaceutical composition” can refer to a preparation of one or more of the active ingredients or agents described herein, e.g., an autophagy promoter and a diuretic with other components, such as physiologically suitable carriers and excipients. The purpose of a pharmaceutical composition is to facilitate administration of one or a combination of active ingredients or agents to a subject.

Compositions

One aspect of the present application can include a composition for preventing or treating skin damage in the skin of mammalian subject in need thereof. The composition can comprise at least one autophagy promoter and at least one diuretic in combination with the autophagy promoter. The at least one autophagy promoter and the at least one diuretic can each be present in an amount to prevent or treat the skin damage.

In some instances, the autophagy promoter can be present in the composition in an amount sufficient to minimize injury and/or promote healing at the site of skin damage in the subject. Efficacy of the autophagy promoter in minimizing injury and/or promoting healing at the site of skin damage can be determined, for example, by detecting one or a combination of the following: a reduction (relative to a control) in known tissue biomarkers of skin inflammation (e.g., TNF-α, IL-6, IL-8, COX-2, and iNOS) as assayed by RT-PCR, for instance; a reduction (relative to a control) in redness/erythema (e.g., as assayed or detected by a chromameter); a reduction (relative to a control) in any one or combination of skin thickness, skin swelling, induration, edema, necrosis, apoptosis, vesication, and macrophage cutaneous infiltration as assayed or measured by histopathology (including using hematoxylin and eosin stains); and a reduction (relative to a control) of any one or combination of pain, irritation and itch, as measured, for example, by a subject through a self-monitoring patient report outcomes device (e.g., PRODIARY, CamNtech, Inc., Boerne, Tex.) The diuretic can be present in the composition in an amount sufficient to reduce or prevent swelling at the site of the skin damage.

In other instances, the at least on diuretic can be present in an amount sufficient to reduce or prevent swelling at the site of skin damage.

In still other instances, the at least one autophagy promoter and the at least one diuretic can each be present in the composition in an amount effective to minimize injury and/or promote healing and/or reduce or prevent swelling at the site of skin damage in the subject.

Compositions of the present application can be formulated differently depending, for example, on the intended route of administration. Non-limiting examples of routes of administration can include oral, inhalation, sublingual, injection (e.g., intravenous or intraperitoneal), or topical.

In another aspect, the autophagy promoter can include an mTOR pathway inhibitor (e.g., rapamycin,), Vitamin D, a Vitamin D analogue, a pharmaceutically active source of Vitamin D, or an mTOR pathway-independent agent (e.g., trehalose).

In one example, the autophagy promoter can include Vitamin D. The term “vitamin D” can refer to the secosterols ergocalciferol (vitamin D₂) and cholecalciferol (vitamin D₃) as well as to their metabolites and analogs, including alfacalcidol (lhydroxycholecalciferol), calcitriol (1α,25-dihydroxycholecalciferol) and dihydrotachysterol. Vitamin D analogs are described in U.S. Pat. No. 4,851,401 (cyclopentano-vitamin D analogs), U.S. Pat. No. 5,120,722 (trihydroxycalciferol derivatives), U.S. Pat. No. 5,446,035 (20-methyl substituted vitamin D), U.S. Pat. No. 5,411,949 (23-oxa-derivatives), U.S. Pat. No. 5,237,110 (19-nor-vitamin D compounds), U.S. Pat. No. 4,857,518 (hydroxylated 24-homo-vitamin D derivatives). Additional Vitamin D analogs are taught in U.S. Pat. Nos. 4,804,502; 4,866,048; 5,145,846 5,374,629; 5,403,940; 5,446,034; and 5,447,924, among others.

In another aspect, the diuretic can include a high ceiling/loop diuretic, a thiazide, a carbonic anhydrase inhibitor, a potassium-sparing diuretic, a calcium-sparing diuretic, an osmotic diuretic, a low ceiling diuretic, a xanthine diuretic, or a diuretic of another mechanism. For example, the diuretic can be spironolactone, furosemide, hydrochlorothiazide, pamabrom, caffeine, or stevia.

In another aspect, active agents of the present application (e.g., autophagy promoters and diuretics) can be formulated as a pharmaceutical composition. Such pharmaceutical compositions can be formulated with other components, such as physiologically suitable carriers and excipients. A carrier (or diluent) can include any agent, compound, or moiety that does not cause significant irritation to a subject and does not abrogate the biological activity and properties of the administered active agents. Examples of acceptable carriers that are useful in the context of the present application include, without limitation, emulsions, creams, aqueous solutions, oils, ointments, pastes, gels, lotions, milks, foams, suspensions and powders. Acceptable carriers can further include, for example, a thickener, an emollient, an emulsifier, a humectant, a surfactant, a suspending agent, a film forming agent, a foam building agent, a preservative, an antifoaming agent, a fragrance, a lower monoalcoholic polyol, a high boiling point solvent, a propellant, a colorant, a pigment or mixtures thereof.

Excipients that may be used to formulate the compositions of the present application can include an inert substance that facilitates administration of the active ingredients. Examples, without limitation, of excipients include calcium carbonate, calcium phosphate, various sugars and types of starch, cellulose derivatives, gelatin, vegetable oils, and polyethylene glycols.

The compositions of the present application may be manufactured by processes well known in the art, e.g., by means of conventional mixing, dissolving, granulating, dragger-making, levigating, emulsifying, encapsulating, entrapping or lyophilizing processes.

Methods

Another aspect of the present application can include a method for preventing or treating skin damage in the skin of a mammalian subject (e.g., a human) in need thereof. “A mammalian subject in need thereof” can include a subject that is at risk of skin damage, a subject that is suspected of having skin damage, or a subject that is already suffering from skin damage. The method can comprise administering a therapeutically effective amount of a composition to the subject, thereby preventing or treating the skin damage. The composition can include at least two active ingredients, e.g., at least one autophagy promoter and at least one diuretic, which are formulated as a pharmaceutical composition.

Depending on the subject, the site (or sites) of skin damage (or suspected skin damage), the composition can be administered via an effective route. Examples of effective administration routes are described above.

The composition can be administered to the subject by the subject himself or herself, or by another person, e.g., a healthcare provider. The composition can be administered according to a prescribed treatment protocol (e.g., as determined by a healthcare professional) or as needed by a subject. In one example, skin damage can be reduced in the subject by at least 5% or more, at least 10% or more, at least 20% or more, at least 25% or more, at least 30% or more, at least 35% or more, at least 40% or more, at least 45% or more, at least 50% or more, at least 55% or more, at least 60% or more, at least 65% or more, at least 70% or more, at least 75% or more, at least 80% or more, at least 85% or more, at least 90% or more, at least 95% or more, or entirely (100%).

In some instances, a reduction in skin damage can be assessed based on the measured induction of repithelialization (i.e., the generation of new cells in the epithelium) or the measured decrease in wound area.

Another aspect of the present disclosure can include a method for preventing or treating skin damage in skin of a mammalian subject in need thereof. A first step of the method can include administering, to the subject, a therapeutically effective amount of a composition comprising at least one autophagy promoter and at least one diuretic. In some instances, the composition can be formulated for oral administration to the subject, e.g., as a liquid, syrup, or a pill (e.g., a gel tab). In one example, the at least one autophagy promoter can comprise vitamin D and the at least one diuretic can comprise spironolactone. Other examples of suitable autophagy promoters and diuretics are described above. In one example, vitamin D can be present in the composition in an amount of about 50,000 I.U. to about 300,000 I.U., e.g., about 100,000 I.U. to about 200,000 I.U. In another example, spironolactone can be present in the composition in an amount of about 50 mg to about 500 mg, e.g., about 100 mg to about 400 mg. The composition can be administered to the subject in the absence or presence of a medical professional, e.g., after the subject and/or a medical professional believes that the subject is in need of skin damage prevention or treatment.

A second step of the method can include administering to the subject, after a period of time following the first step, a therapeutically effect amount of a series of diuretic compositions. In one example, each of the diuretic compositions comprising the series of diuretic compositions can include spironolactone. The amount and/or type of the diuretic comprising each of the diuretic composition can be the same or different. In one example, each of the diuretic compositions comprising the series of diuretic compositions can include about 50 mg to about 300 mg, e.g., about 100 mg to about 200 mg of spironolactone. The period of time can include at least 12 hours, e.g., at least 24 hours or more. In some instances, each diuretic composition comprising the series of diuretic compositions can be separately administered to the subject at consecutive time points following the first step of the method. For example, a first diuretic composition can be administered one day after the first step, a second diuretic composition can be administered one day after the first diuretic composition is administered, and a third diuretic composition can be administered one day after the second diuretic composition is administered to the subject.

In one example of the method, a first step of the method can include administering, to a subject in need thereof, a therapeutically effective amount of a composition comprising about 100,000 to about 200,000 I.U. vitamin D and about 100 mg to about 400 mg spironolactone. The composition can be formulated for oral administration to the subject (e.g., as a pill or gel-tab). After the subject has ingested the composition, a series of three diuretic compositions can be separately administered to the subject at different, consecutive time points. Each of the diuretic compositions can comprise about 100 mg to about 200 mg of spironolactone and be formulated for oral administration (e.g., as a pill or gel-tab). A first diuretic composition comprising the series of three diuretic compositions can be administered about 24 hours after the composition (comprising vitamin D and spironolactone) has been administered, a second diuretic composition comprising the series of three diuretic compositions can be administered about 24 hours after the first diuretic composition, and a third diuretic composition comprising the series of three diuretic compositions can be administered about 24 hours after the second diuretic composition.

Advantageously, administering at least one autophagy agent (e.g., vitamin D) and a diuretic (e.g., spironolactone) as a one-time treatment, followed by daily administration of a diuretic (e.g., spironolactone), can reduce skin inflammation, prevent skin scab (necrosis), heal and reduce wound size, and reduce skin swelling.

Another aspect of the present disclosure can include a kit for preventing or treating skin damage in skin of a mammalian subject in need thereof. The kit can comprise a therapeutically effective amount of a composition comprising at least one autophagy promoter and at least one diuretic. In some instances, the composition can be formulated for oral administration to the subject, e.g., as a liquid, syrup, or a pill (e.g., a gel tab). In one example, the at least one autophagy promoter can comprise vitamin D and the at least one diuretic can comprise spironolactone. Other examples of suitable autophagy promoters and diuretics are described above. In one example, vitamin D can be present in the composition in an amount of about 50,000 I.U. to about 300,000 I.U., e.g., about 100,000 I.U. to about 200,000 I.U. In another example, spironolactone can be present in the composition in an amount of about 50 mg to about 500 mg, e.g., about 100 mg to about 400 mg.

The kit can further comprise a series of diuretic compositions, each of which is formulated for oral administration e.g., as a liquid, syrup, or a pill (e.g., a gel tab). In one example, each of the diuretic compositions comprising the series of diuretic compositions can include spironolactone. The amount and/or type of the diuretic in each of the diuretic composition can be the same or different. In one example, each of the diuretic compositions comprising the series of diuretic compositions can include about 50 mg to about 300 mg, e.g., about 100 mg to about 200 mg of spironolactone.

The kit can also include a set of dosing instructions. The instructions can provide a subject with appropriate directions for using the kit and its components. For example, the instructions can include the following set of directions: “(1) ingest the composition (comprising at least one autophagy promoter and at least one diuretic) on a first day; and (2) subsequently ingest each of the diuretic compositions on separate consecutive days”. By way of further example, a first diuretic composition comprising the series of three diuretic compositions can be ingested about 24 hours after the composition (comprising vitamin D and spironolactone) has been ingested, a second diuretic composition comprising the series of three diuretic compositions can be ingested about 24 hours after the first diuretic composition, and a third diuretic composition comprising the series of three diuretic compositions can be ingested about 24 hours after the second diuretic composition.

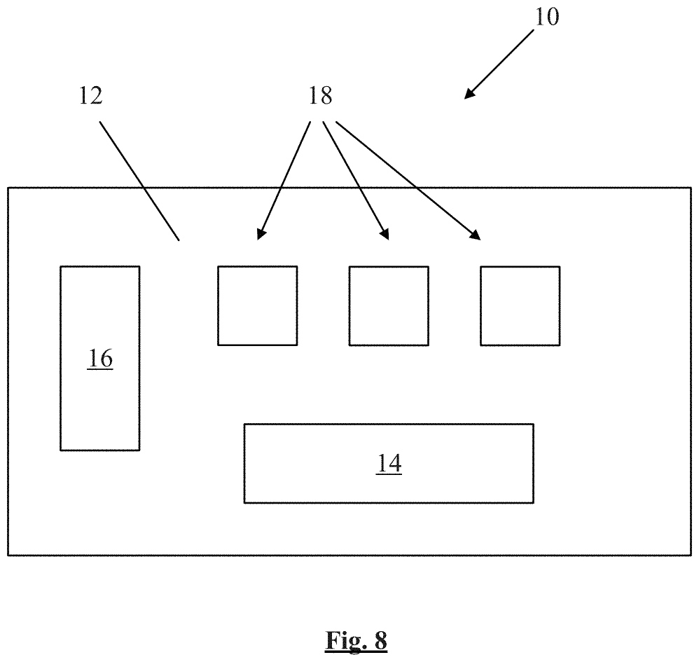

The components of the kit can be packaged for easy and convenient use by a subject. One example of a kit 10 according to the present disclosure is shown in FIG. 8. The kit 10 can comprise a blister pack or other similar packaging (e.g., similar to a “Z-pack”) configuration. In this configuration, the kit 10 can comprise an outer packaging material (e.g., a cardboard or plastic box) 12, a set of instructions 14 either disposed within the packaging material or printed thereon, a first blister container 16 for retaining the composition (comprising at least one autophagy promoter and at least one diuretic) (e.g., where the composition is formulated as a pill or gel-tab), and a series of blister containers 18 for retaining each of the diuretic compositions (e.g., where each of the diuretic compositions is formulated as a pill or gel-tab).

The following examples are for the purpose of illustration only and are not intended to limit the scope of the claims, which are appended hereto.

### Example 1

Tissue injury from burns or exposure to toxicants initiates a series of reactions that call into action release of inflammatory mediators at the site of injury. In response, vascular leakage prompts influx of inflammatory cells including macrophages to the site of injury evolving into a microenvironment with increased osmolarity and high Na+ concentration. While increased Na+ concentration supports inflammation and cellular activation with upregulation of anti-microbial defenses in skin, an increase in osmolarity in the injured tissue microenvironment prevents the natural turnover of infiltrating macrophages by doubling their half-life through down regulation of anti-apoptotic factors and augmenting macrophage polarization to the activated M1 inflammatory phenotype. The mineralocorticoid receptor (MR) antagonist spironolactone (SP) inhibits macrophage polarization and functions as a diuretic making it an ideal candidate for treatment of reducing NM-induced skin edema and macrophage activation.

We hereby propose that SP being an MR antagonist and a weak diuretic has sufficient capacity to reduce water content of mustard-induced edematous skin lesions as a way to accelerate wound healing. Treatment of NM-challenged mice with 25(OH)D in conjunction with SP may prove to be a novel two-pronged approach to (a) reduce edema to accelerate wound healing and (b) reduce inflammation by dampening the innate immune response that is triggered by skin injury.

Mustard injured skin that is characterized by skin vesication, macrophage-mediated inflammation, and induration can be countered by treatment with a diuretic to decrease Na+ concentration as a way of reducing edema and prevent macrophage activation. SP is a potassium-sparing diuretic (water pill) that has been used to treat edema in patients with hypertension, congestive heart failure, lung injury and other kidney pathologies. At the molecular level, SP is a synthetic steroid that acts as a competitive antagonist of the corticosteroid aldosterone. While much information has been accumulated on the pathophysiology of MR cellular and molecular targets, the effect of MR ligands and blockers on non-classical targets, such as in skin pathology have led to new and exciting potential application. Additionally, there is some evidence that SP ameliorates diabetic pain via reducing inflammatory molecules in the spleen and that it mitigates collagen deposition and scarring leading to accelerated healing in a rat model of myocardial infarction.

### Determine Whether Reducing Local Skin Pathology Accelerates NM-Mediated Wound Healing and Confers Systemic Protection

Attenuation of Swelling and Macrophage Activation with SP Treatment to Alleviate pain and skin destruction

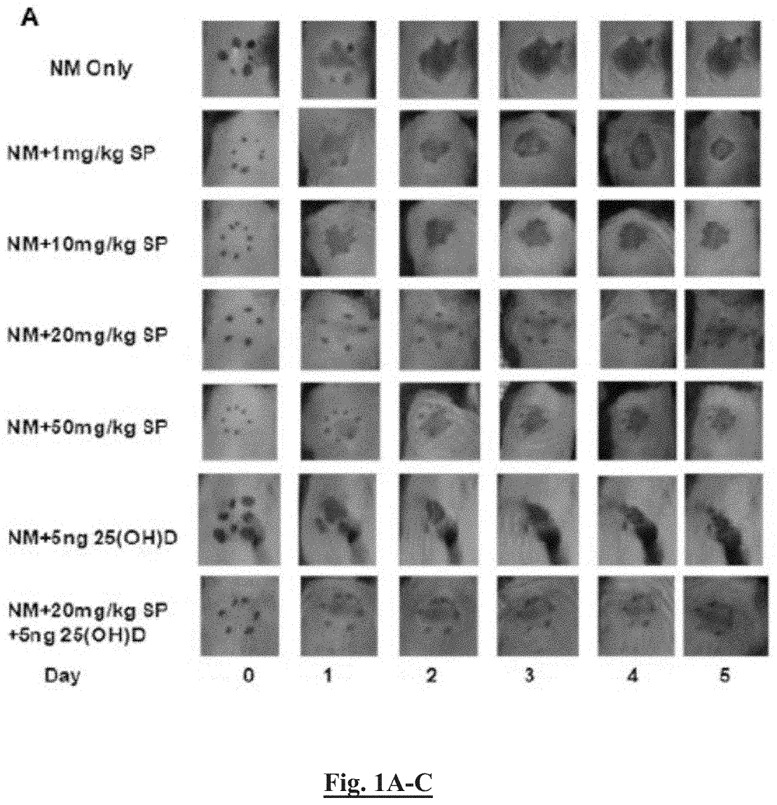

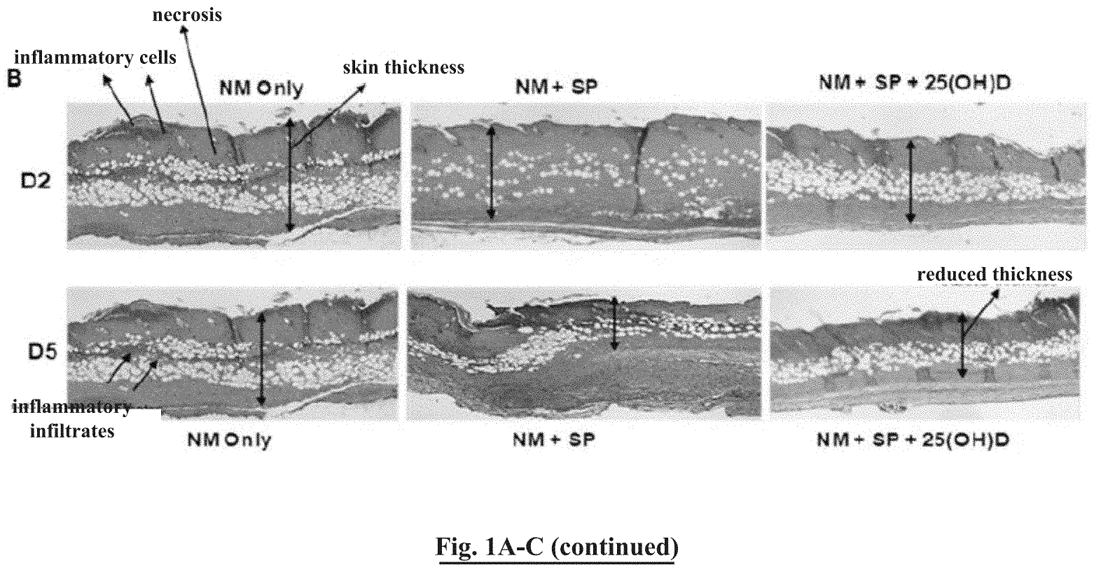

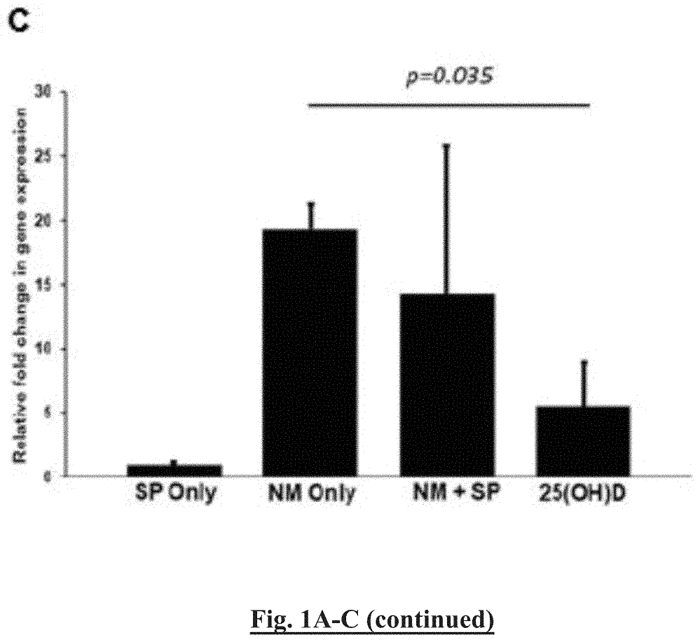

Mice challenged with NM showed a dose dependent reduction in lesion size upon treatment with SP. Although 50 mg/kg SP did reduce lesion area at day 5, the mice sustained systemic toxicity leading us to select a lower dose of 20 mg/kg SP to act in conjunction with 25(OH)D (FIG. 1A). A dramatic difference in skin thickness is observed from histological images as early as 48 h post NM challenge in presence of SP and 25(OH)D.

While NM-challenged mouse exhibits skin vesication, near complete erosion of the epidermal layer while the dermis is thickly populated with inflammatory cells contributing to dramatic increase in skin thickness, the SP treated skin showed significant reduction in inflammatory cells as early as day 2 and decrease in thickness by day 5. Best results are shown with combined treatment including SP and 25(OH)D (FIG. 1B). Although studies showed that SP contributes to decreasing inflammatory markers like iNOS, in our hands, iNOS levels responded to 25(OH)D treatment alone (FIG. 1C) focusing our efforts to use SP to reduce skin swelling at the site of injury.

Determine Whether Reduction in Edema in NM-Mediated Skin Injury by Treatment with SP Alone or in Combination with Vitamin D Offers Protection Local and Systemic Pathology

Mice exposed to NM are subjected to 200 ul i.p. injection with SP (Sigma-Aldrich) in the range of 20-50 mg/kg. Following NM challenge, wound area and skin thickness are measured to evaluate efficacy of SP in reducing edema and how in combination with 25(OH)D, both local and systemic pathology from NM exposure are addressed. Skin biopsies are obtained at days 0 through 5 following NM challenge. In addition to measuring inflammatory markers iNOS and TNFα by qRT-PCR (TaqMan Gene Expression, Applied Biosystems), markers for pain such as TRPv143 from skin biopsies are also evaluated. Additional skin specimens are embedded and sectioned for microscopy and histopathology. Each animal has a day “0” biopsy of normal unexposed skin as paired controls. The degree of macrophage infiltration, amount of TNFα and iNOS protein and co-localization between cells and signaling molecules are assessed using confocal microscopy and Metamorph analysis (software version 7.1 MDS Analytical Tech., Sunnyvale, Calif.). Macros are tailored to our project enabling the software to quantify positive fluorescence staining of 6 skin sections visualized at 20× magnification. For administering SP to mice, a dose of 20 mg/kg SP is diluted in 20% alcohol and introduced in the mice 1 h after NM exposure as done with 25(OH)D. The time consideration takes into account recovery from general anesthesia and intubation (for NM application) with demonstration of ability to eat and drink. Biomarker analysis (iNOS and TNFα) obtained in the initial study is used to perform sample size calculations. Outcomes and values are compared to treatment with oral cholecalciferol D3 alone.

Survival Studies

Survival studies are conducted only in the murine model. The criteria include animals found dead or meeting the cut-off for compassionate euthanasia (>25% loss in body weights). Based on preliminary data, sample size calculations predict that a sample size of 13 animals per group provides 79% power to detect the observed difference in mean weight loss, while 14 per group provides 82% power. A sample size of 16 per group provides sufficient power to detect differences in survival rates as well as comparisons of mean weight loss at specific time points in the current murine model. SP is given at 1 hr after NM exposure. Indicators of morbidity such as weight loss are used; as such daily weight measurements are performed.

Methods for Analysis of Hematological Specimens

In our current NM mouse model, by day 5, there are approximately 75% fewer cells in the bone marrow. Flow cytometric analysis of the hematopoietic populations (from femurs) shows a dramatic loss of the lymphoid and erythroid populations. This correlates with CBCs showing marked anemia and lymphopenia done at the Mouse Physiology Phenotyping Center at Case Western Reserve University.

We expect that combination treatment with SP and 25(OH)D following NM challenge on skin mitigates early inflammatory influx of macrophages in response to chemotactic mediators at the site of injury. Additionally, the swelling and pain markers are expected to reduce as an overall improvement of skin wound healing. Local attenuation of macrophage activation is expected to attenuate the long term deleterious systemic effects of NM on bone marrow and blood.

### Example 2

Intervention with Loop Diuretic Monotherapy and Combination Treatment with Vitamin D

An experiment is performed in which the effects of monotherapy with a loop diuretic (e.g., furosemide) and combination therapy with vitamin D are studied. Using the NM mouse skin wound model in Example 1, two groups of mice (each group n=3) with NM-challenged skin are exposed to either furosemide (e.g., at a desired dosage in mg/kg) alone or in combination with 25OHD (e.g., at a desired dosage in mg/kg). Daily wound progression after topical exposure to NM is evaluated for each group. Relative iNOS gene expression 48 hours post NM challenge is also assessed for each group.

Determine Whether Reduction in Edema in NM-Mediated Skin Injury by Treatment with the Loop Diuretic Alone or in Combination with Vitamin D Offers Protection Local and Systemic Pathology

Mice exposed to NM are subjected to 200 ul i.p. injection with furosemide (e.g., at a desired dosage in mg/kg). Following NM challenge, wound area and skin thickness are measured to evaluate efficacy of furosemide in reducing edema and how in combination with 25(OH)D, both local and systemic pathology from NM exposure are addressed. Skin biopsies are obtained at days 0 through 5 following NM challenge. In addition to measuring inflammatory markers iNOS and TNFα by qRT-PCR (TaqMan Gene Expression, Applied Biosystems), markers for pain such as TRPv143 from skin biopsies are also evaluated. Additional skin specimens are embedded and sectioned for microscopy and histopathology. Each animal has a day “0” biopsy of normal unexposed skin as paired controls. The degree of macrophage infiltration, amount of TNFα and iNOS protein and co-localization between cells and signaling molecules are assessed using confocal microscopy and Metamorph analysis (software version 7.1 MDS Analytical Tech., Sunnyvale, Calif.). Macros are tailored to our project enabling the software to quantify positive fluorescence staining of 6 skin sections visualized at 20× magnification. The time consideration takes into account recovery from general anesthesia and intubation (for NM application) with demonstration of ability to eat and drink. Biomarker analysis (iNOS and TNFα) obtained in the initial study is used to perform sample size calculations. Outcomes and values are compared to treatment with oral cholecalciferol D3 alone. Survival studies and methods for analysis of hematological specimens are done as in Example 1.

We expect that combination treatment with a loop diuretic (e.g., furosemide) and 25(OH)D following NM challenge on skin mitigates early inflammatory influx of macrophages in response to chemotactic mediators at the site of injury. Additionally, the swelling and pain markers are expected to reduce as an overall improvement of skin wound healing.

### Example 3

Intervention with Thiazide Diuretic Monotherapy and Combination Treatment with Vitamin D

An experiment is performed in which the effects of monotherapy with a thiazide diuretic (e.g., hydrochlorothiazide) and combination therapy with vitamin D are studied. Using the NM mouse skin wound model in Example 1, two groups of mice (each group n=3) with NM-challenged skin are exposed to either hydrochlorothiazide (e.g., at a desired dosage in mg/kg) alone or in combination with 25(OH)D (e.g., at a desired dosage in mg/kg). Daily wound progression after topical exposure to NM is evaluated for each group. Relative iNOS gene expression 48 hours post NM challenge is also assessed for each group.

Determine Whether Reduction in Edema in NM Mediated Skin Injury by Treatment with the Thiazide Diuretic Alone or in Combination with Vitamin D Offers Protection Local and Systemic Pathology

Mice exposed to NM are subjected to 200 ul i.p. injection with hydrochlorothiazide (e.g., at a desired dosage in mg/kg). Following NM challenge, wound area and skin thickness are measured to evaluate efficacy of hydrochlorothiazide in reducing edema and how in combination with 25(OH)D, both local and systemic pathology from NM exposure are addressed. Skin biopsies are obtained at days 0 through 5 following NM challenge. In addition to measuring inflammatory markers iNOS and TNFα by qRT-PCR (TaqMan Gene Expression, Applied Biosystems), markers for pain such as TRPv143 from skin biopsies are also evaluated. Additional skin specimens are embedded and sectioned for microscopy and histopathology. Each animal has a day “0” biopsy of normal unexposed skin as paired controls. The degree of macrophage infiltration, amount of TNFα and iNOS protein and co-localization between cells and signaling molecules are assessed using confocal microscopy and Metamorph analysis (software version 7.1 MDS Analytical Tech., Sunnyvale, Calif.). Macros are tailored to our project enabling the software to quantify positive fluorescence staining of 6 skin sections visualized at 20× magnification. The time consideration takes into account recovery from general anesthesia and intubation (for NM application) with demonstration of ability to eat and drink. Biomarker analysis (iNOS and TNFα) obtained in the initial study is used to perform sample size calculations. Outcomes and values are compared to treatment with oral cholecalciferol D3 alone. Survival studies and methods for analysis of hematological specimens are done as in Example 1.

We expect that combination treatment with a thiazide diuretic (e.g., hydrochlorothiazide) and 25(OH)D following NM challenge on skin mitigates early inflammatory influx of macrophages in response to chemotactic mediators at the site of injury. Additionally, the swelling and pain markers are expected to reduce as an overall improvement of skin wound healing.

### Example 4

Intervention with Spironolactone (SP) Monotherapy and Combination Treatment with an AMPK Activator

An experiment is performed in which the effects of monotherapy with SP and combination therapy with an AMPK activator (e.g., metformin) are studied. Using the NM mouse skin wound model in Example 1, two groups of mice (each group n=3) with NM-challenged skin are exposed to either SP (e.g., at a desired dosage in mg/kg) alone or in combination with metformin (e.g., at a desired dosage in mg/kg). Daily wound progression after topical exposure to NM is evaluated for each group. Relative iNOS gene expression 48 hours post NM challenge is also assessed for each group.

Determine Whether Reduction in Edema in NM-Mediated Skin Injury by Treatment with SP Alone or in Combination with the AMPK Activator Offers Protection Local and Systemic Pathology

Mice exposed to NM are subjected to 200 ul i.p. injection with SP (e.g., at a desired dosage in mg/kg). Following NM challenge, wound area and skin thickness are measured to evaluate efficacy of SP in reducing edema and how in combination with an AMPK activator (e.g., metformin), both local and systemic pathology from NM exposure are addressed. Skin biopsies are obtained at days 0 through 5 following NM challenge. In addition to measuring inflammatory markers iNOS and TNFα by qRT-PCR (TaqMan Gene Expression, Applied Biosystems), markers for pain such as TRPv143 from skin biopsies are also evaluated. Additional skin specimens are embedded and sectioned for microscopy and histopathology. Each animal has a day “0” biopsy of normal unexposed skin as paired controls. The degree of macrophage infiltration, amount of TNFα and iNOS protein and co-localization between cells and signaling molecules are assessed using confocal microscopy and Metamorph analysis (software version 7.1 MDS Analytical Tech., Sunnyvale, Calif.). Macros are tailored to our project enabling the software to quantify positive fluorescence staining of 6 skin sections visualized at 20× magnification. The time consideration takes into account recovery from general anesthesia and intubation (for NM application) with demonstration of ability to eat and drink. Biomarker analysis (iNOS and TNFα) obtained in the initial study is used to perform sample size calculations. Outcomes and values are compared to treatment with oral metformin alone. Survival studies and methods for analysis of hematological specimens are done as in Example 1.

We expect that combination treatment with SP and an AMPK inhibitor (e.g., metformin) following NM challenge on skin mitigates early inflammatory influx of macrophages in response to chemotactic mediators at the site of injury. Additionally, the swelling and pain markers are expected to reduce as an overall improvement of skin wound healing.

### Example 5

Intervention with Spironolactone (SP) Monotherapy and Combination Treatment with an mTOR Inhibitor

An experiment is performed in which the effects of monotherapy with SP and combination therapy with an mTOR inhibitor (e.g., rapamycin) are studied. Using the NM mouse skin wound model in Example 1, two groups of mice (each group n=3) with NM-challenged skin are exposed to either SP (e.g., at a desired dosage in mg/kg) alone or in combination with rapamycin (e.g., at a desired dosage in mg/kg). Daily wound progression after topical exposure to NM is evaluated for each group. Relative iNOS gene expression 48 hours post NM challenge is also assessed for each group.

Determine Whether Reduction in Edema in NM-Mediated Skin Injury by Treatment with SP Alone or in Combination with the mTOR Inhibitor Offers Protection Local and Systemic Pathology

Mice exposed to NM are subjected to 200 ul i.p. injection with SP (e.g., at a desired dosage in mg/kg). Following NM challenge, wound area and skin thickness are measured to evaluate efficacy of SP in reducing edema and how in combination with an mTOR inhibitor (e.g., rapamycin), both local and systemic pathology from NM exposure are addressed. Skin biopsies are obtained at days 0 through 5 following NM challenge. In addition to measuring inflammatory markers iNOS and TNFα by qRT-PCR (TaqMan Gene Expression, Applied Biosystems), markers for pain such as TRPv143 from skin biopsies are also evaluated. Additional skin specimens are embedded and sectioned for microscopy and histopathology. Each animal has a day “0” biopsy of normal unexposed skin as paired controls. The degree of macrophage infiltration, amount of TNFα and iNOS protein and co-localization between cells and signaling molecules are assessed using confocal microscopy and Metamorph analysis (software version 7.1 MDS Analytical Tech., Sunnyvale, Calif.). Macros are tailored to our project enabling the software to quantify positive fluorescence staining of 6 skin sections visualized at 20× magnification. The time consideration takes into account recovery from general anesthesia and intubation (for NM application) with demonstration of ability to eat and drink. Biomarker analysis (iNOS and TNFα) obtained in the initial study is used to perform sample size calculations. Outcomes and values are compared to treatment with oral rapamycin alone. Survival studies and methods for analysis of hematological specimens are done as in Example 1.

We expect that combination treatment with SP and an mTOR inhibitor (e.g., rapamycin) following NM challenge on skin mitigates early inflammatory influx of macrophages in response to chemotactic mediators at the site of injury. Additionally, the swelling and pain markers are expected to reduce as an overall improvement of skin wound healing.

### Example 6

Determine Whether Reduction in Edema in UV-Mediated Skin Injury by Treatment with SP Alone or in Combination with Vitamin D Offers Protection Against Local and Systemic Pathology

An experiment is performed in which the effects of monotherapy with SP and combination therapy with vitamin D are studied. Using a UV skin injury model (described below), two groups of human subjects (each group n=5) with UV-mediated skin injury (1×, 2× and 3×MED UV radiation) are exposed to either SP (e.g., at a desired dosage in mg/kg) alone or in combination with vitamin D (e.g., at a desired dosage in mg/kg). Chromameter readings at 24 and 48 hours and skin thickness at 72 hours and 1 week post administration of SP and combination therapy are performed. Relative iNOS and TNF-α gene expression 48 hours post UV exposure is also assessed for each group.

We expect that combination treatment with SP and vitamin D following UV radiation exposure on skin mitigates early inflammatory influx of macrophages in response to chemotactic mediators at the site of injury. Additionally, the swelling and pain markers are expected to reduce as an overall improvement of skin wound healing.

The UV Injury Model

The UV skin injury study (institutional review board (IRB) approved) was conducted as a randomized double-blinded placebo controlled trial of 20 patients with the goal to determine the optimal dose of vitamin D that is adequate to suppress (a) skin redness by chromameter analysis and digital imaging (b) skin swelling by digital calipers (c) histologic evaluation of vesication of skin biopsies (d) qRT-PCR of inflammatory factors TNFα and iNOS from skin biopsies. All patients were evaluated by inclusion and exclusion criteria and were evaluated to exclude oral vitamin D and NSAIDS use. All patients had blood samples taken at baseline and at subsequent visits to test for vitamin D levels. All patients were subjected to UV radiation testing to determine their individual sunburn dose, also known as the “minimal erythema dose” (MED) of solar radiation that would result in an incremental increase in redness (fitted on linear regression curve) in the skin measured by chromameter readings. The subjects were then exposed to 1× (mild), 2× (moderate), and 3× (severe) their MED dose on the left inner arm. An additional 3×MED site was performed so that a skin biopsy could be procured at 48 hours for cellular and molecular studies. One week following the tests, patients returned for repeat of the same protocol on the contralateral (right) arm. The only addition was that 1 hour after the UV exposure, patients were given intervention drug or placebo. The medications were randomized and blinded by our institution's investigational pharmacy. A study assistant witnesses patients orally ingesting the medication. As a result, each patient represents their own “paired control” so that comparisons can be made. In total, all patients received UV radiation, 40 “paired” biopsies specimens obtained. All patients had serum collected at days 0, 1, 2, 3, and day 7 after ingestion of vitamin D or placebo. In summary, 20 patients completed the trial with each patient having 13 visits to our skin study center. The breakdown is as follows: 6 patients—received placebo; 5 received 50,000 I.U. vitamin D3; 4 received 100,000 I.U. vitamin D3; and 5 received 200,000 I.U. vitamin D3.

### Example 7

Determine Whether Reduction in Edema in UV-Mediated Skin Injury by Treatment with a Loop Diuretic Alone or in Combination with Vitamin D Offers Protection Against Local and Systemic Pathology

An experiment is performed in which the effects of monotherapy with a loop diuretic (e.g., furosemide) and combination therapy with vitamin D are studied. Using a UV skin injury model (described in Example 6), two groups of human subjects (each group n=5) with UV-mediated skin injury (1×, 2× and 3×MED UV radiation) are exposed to either furosemide (e.g., at a desired dosage in mg/kg) alone or in combination with vitamin D (e.g., at a desired dosage in mg/kg). Chromameter readings at 24 and 48 hours and skin thickness at 72 hours and 1 week post administration of furosemide and combination therapy are performed. Relative iNOS and TNF-α gene expression 48 hours post UV exposure is also assessed for each group.

We expect that combination treatment with a loop diuretic (e.g., furosemide) and vitamin D following UV radiation exposure on skin mitigates early inflammatory influx of macrophages in response to chemotactic mediators at the site of injury. Additionally, the swelling and pain markers are expected to reduce as an overall improvement of skin wound healing.

### Example 8

Determine Whether Reduction in Edema in UV-Mediated Skin Injury by Treatment with a Thiazide Diuretic Alone or in Combination with Vitamin D Offers Protection Against Local and Systemic Pathology

An experiment is performed in which the effects of monotherapy with a thiazide diuretic (e.g., hydrochlorothiazide) and combination therapy with vitamin D are studied. Using a UV skin injury model (described in Example 6), two groups of human subjects (each group n=5) with UV-mediated skin injury (1×, 2× and 3×MED UV radiation) are exposed to either hydrochlorothiazide (e.g., at a desired dosage in mg/kg) alone or in combination with vitamin D (e.g., at a desired dosage in mg/kg). Chromameter readings at 24 and 48 hours and skin thickness at 72 hours and 1 week post administration of hydrochlorothiazide and combination therapy are performed. Relative iNOS and TNF-α gene expression 48 hours post UV exposure is also assessed for each group.

We expect that combination treatment with a thiazide diuretic (e.g., hydrochlorothiazide) and vitamin D following UV radiation exposure on skin mitigates early inflammatory influx of macrophages in response to chemotactic mediators at the site of injury. Additionally, the swelling and pain markers are expected to reduce as an overall improvement of skin wound healing.

### Example 9

Determine Whether Reduction in Edema in UV-Mediated Skin Injury by Treatment with Spironolactone (SP) Alone or in Combination with an AMPK Activator Offers Protection Against Local and Systemic Pathology

An experiment is performed in which the effects of monotherapy with SP and combination therapy with an AMPK activator (e.g., metformin) are studied. Using a UV skin injury model (described in Example 6), two groups of human subjects (each group n=5) with UV-mediated skin injury (1×, 2× and 3×MED UV radiation) are exposed to either SP (e.g., at a desired dosage in mg/kg) alone or in combination with metformin (e.g., at a desired dosage in mg/kg). Chromameter readings at 24 and 48 hours and skin thickness at 72 hours and 1 week post administration of SP and combination therapy are performed. Relative iNOS and TNF-α gene expression 48 hours post UV exposure is also assessed for each group.

We expect that combination treatment with SP and an AMPK activator (e.g., metformin) following UV radiation exposure on skin mitigates early inflammatory influx of macrophages in response to chemotactic mediators at the site of injury. Additionally, the swelling and pain markers are expected to reduce as an overall improvement of skin wound healing.

### Example 10

Determine Whether Reduction in Edema in UV-Mediated Skin Injury by Treatment with Spironolactone (SP) Alone or in Combination with an mTOR Inhibitor Offers Protection Against Local and Systemic Pathology

An experiment is performed in which the effects of monotherapy with SP and combination therapy with an mTOR inhibitor (e.g., rapamycin) are studied. Using a UV skin injury model (described in Example 6), two groups of human subjects (each group n=5) with UV-mediated skin injury (1×, 2× and 3×MED UV radiation) are exposed to either SP (e.g., at a desired dosage in mg/kg) alone or in combination with rapamycin (e.g., at a desired dosage in mg/kg). Chromameter readings at 24 and 48 hours and skin thickness at 72 hours and 1 week post administration of SP and combination therapy are performed. Relative iNOS and TNF-α gene expression 48 hours post UV exposure is also assessed for each group.

We expect that combination treatment with SP and an mTOR inhibitor (e.g., rapamycin) following UV radiation exposure on skin mitigates early inflammatory influx of macrophages in response to chemotactic mediators at the site of injury. Additionally, the swelling and pain markers are expected to reduce as an overall improvement of skin wound healing.

### Example 11

Intervention with Xanthine Diuretic Monotherapy and Combination Treatment with Cholecalciferol

An experiment is performed in which the effects of monotherapy with a xanthine diuretic (e.g., pamabrom) and combination therapy with cholecalciferol are studied. Using the NM mouse skin wound model in Example 1, two groups of mice (each group n=3) with NM-challenged skin are exposed to either pamabrom (e.g., at a desired dosage in mg/kg) alone or in combination with cholecalciferol (e.g., at a desired dosage in mg/kg). Daily wound progression after topical exposure to NM is evaluated for each group. Relative iNOS gene expression 48 hours post NM challenge is also assessed for each group.

Determine Whether Reduction in Edema in NM-Mediated Skin Injury by Treatment with the Xanthine Diuretic Alone or in Combination with Cholecalciferol Offers Protection Local and Systemic Pathology

Mice exposed to NM are subjected to 200 ul i.p. injection with pamabrom (e.g., at a desired dosage in mg/kg). Following NM challenge, wound area and skin thickness are measured to evaluate efficacy of furosemide in reducing edema and how in combination with cholecalciferol, both local and systemic pathology from NM exposure are addressed. Skin biopsies are obtained at days 0 through 5 following NM challenge. In addition to measuring inflammatory markers iNOS and TNFα by qRT-PCR (TaqMan Gene Expression, Applied Biosystems), markers for pain such as TRPv143 from skin biopsies are also evaluated. Additional skin specimens are embedded and sectioned for microscopy and histopathology. Each animal has a day “0” biopsy of normal unexposed skin as paired controls. The degree of macrophage infiltration, amount of TNFα and iNOS protein and co-localization between cells and signaling molecules are assessed using confocal microscopy and Metamorph analysis (software version 7.1 MDS Analytical Tech., Sunnyvale, Calif.). Macros are tailored to our project enabling the software to quantify positive fluorescence staining of 6 skin sections visualized at 20× magnification. The time consideration takes into account recovery from general anesthesia and intubation (for NM application) with demonstration of ability to eat and drink. Biomarker analysis (iNOS and TNFα) obtained in the initial study is used to perform sample size calculations. Outcomes and values are compared to treatment with oral cholecalciferol alone. Survival studies and methods for analysis of hematological specimens are done as in Example 1.

We expect that combination treatment with a xanthine diuretic (e.g., pamabrom) and cholecalciferol following NM challenge on skin mitigates early inflammatory influx of macrophages in response to chemotactic mediators at the site of injury. Additionally, the swelling and pain markers are expected to reduce as an overall improvement of skin wound healing

### Example 12

Determine Whether Reduction in Edema in UV-Mediated Skin Injury by Treatment with a Xanthine Diuretic Alone or in Combination with Cholecalciferol Offers Protection Against Local and Systemic Pathology

An experiment is performed in which the effects of monotherapy with a xanthine diuretic (e.g., pamabrom) and combination therapy with cholecalciferol are studied. Using a UV skin injury model (described in Example 6), two groups of human subjects (each group n=5) with UV-mediated skin injury (1×, 2× and 3×MED UV radiation) are exposed to either pamabrom (e.g., at a desired dosage in mg/kg) alone or in combination with cholecalciferol (e.g., at a desired dosage in mg/kg). Chromameter readings at 24 and 48 hours and skin thickness at 72 hours and 1 week post administration of pamabrom and combination therapy are performed. Relative iNOS and TNF-α gene expression 48 hours post UV exposure is also assessed for each group.

We expect that combination treatment with a xanthine diuretic (e.g., pamabrom) and cholecalciferol following UV radiation exposure on skin mitigates early inflammatory influx of macrophages in response to chemotactic mediators at the site of injury. Additionally, the swelling and pain markers are expected to reduce as an overall improvement of skin wound healing.

### Example 13

Methods

Topical Exposure to Skin Irritant/Blistering Agent Nitrogen Mustard

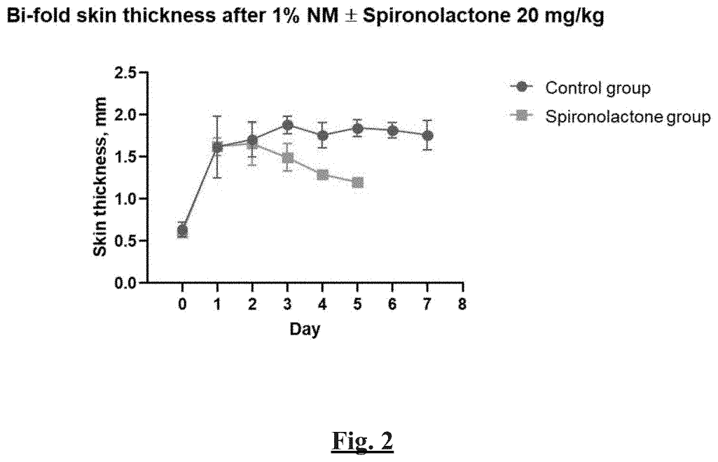

Mice were shaved and depilated on the dorsal back using hair remover cream Nair™. Mice were allowed to rest for a minimum of 3 days before experimental procedure. Mice were exposed topically to an area on the dorsal back (12 mm diameter circle) with a 40 μl solution of dilute 0.5% mechlorethamine (a.k.a. nitrogen mustard, NM) in saline and 1.5% DMSO. 1-2 hours later mice were either treated with 5 ng 25-hydroxyvitamin D3 (herein referred as 25OHD or VD in FIGS. 2-7), spironolactone (SP), combination treatment, or sham vehicle control treatment. 25OHD is given intraperitoneally (i.p.) as a one-time dose. SP in one experiment is given as 20 mg/kg i.p. (dissolved in small aliquot of ethanol and then diluted with normal saline) daily for 3 days. In other experiments SP (200 mg/kg dissolved in water) is given by per orum daily for 3 or 4 days using a flexible oral gavage tube.

Skin Thickness

Skin thickness was measured daily by pinching the skin and measuring of bi-fold skin thickness with digital calipers.

Scab/Wound Area Measurements

Digital photography was obtained of skin area on days 0-5, 7, 14, and 21. Hemorrhagic crusts/scabs were quantified using digital image analysis on imageJ™. Percent scab is obtained by dividing with total initial exposure area size (12 mm circle).

RT-PCR

Quantification of inflammatory genes in the skin was performed from RNA extracted from skin biopsies harvested at necropsy. Genes of interest were assayed using primers, probes, and PCR apparatus from Applied Biosystems™ (standard one-step quantitative RT-PCR).

Results

In FIG. 2, 20 mg/kg of spironolactone showed efficacy in reducing skin thickness. Vitamin D was given on day 0. Spironolactone was given daily for 4 days.

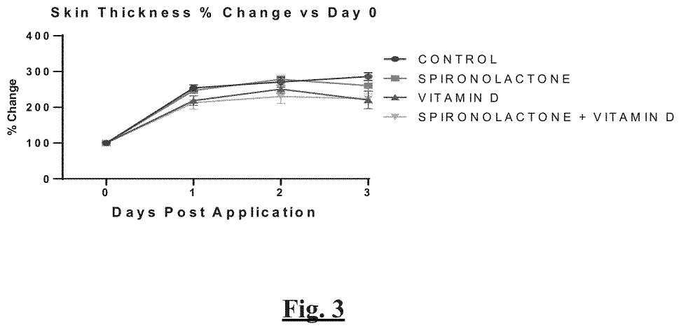

The results in FIG. 3 show that spironolactone+vitamin D combination treatment results in a trend towards reducing skin edema at 24, 48, and 72 hours post-NM exposure. Treatment of animals with spironolactone (daily for 4 days) and vitamin D (day 0) delayed or prevented scab formation following nitrogen mustard exposure.

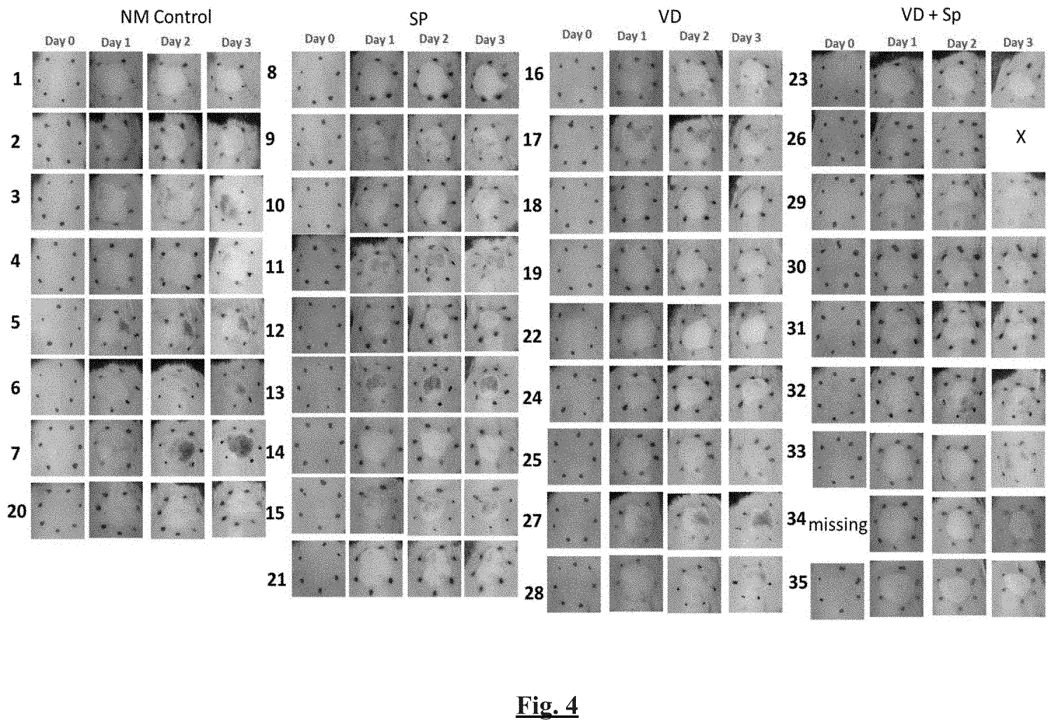

In FIG. 4, we observed a trend of decreased red scab formation in the skin of animals treated with intervention.

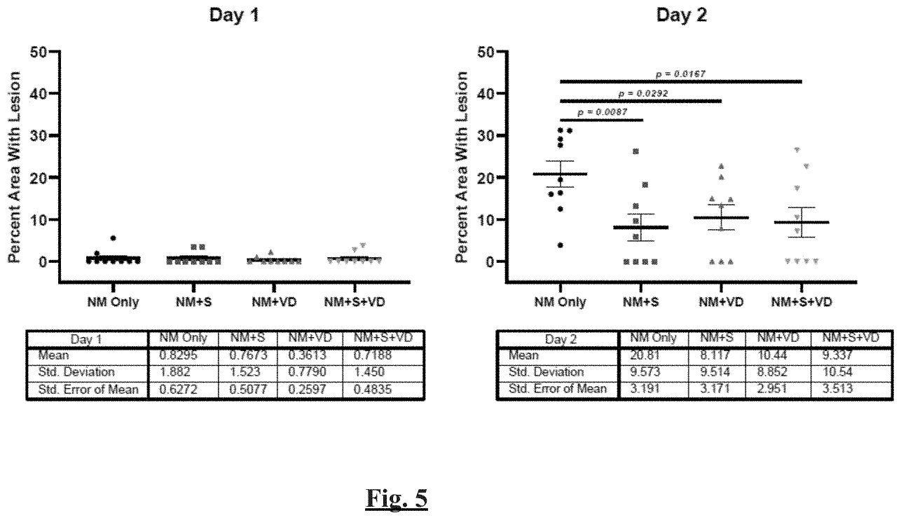

In FIG. 5, the area of scab formation was analyzed by image-J™ software and quantified for all groups.

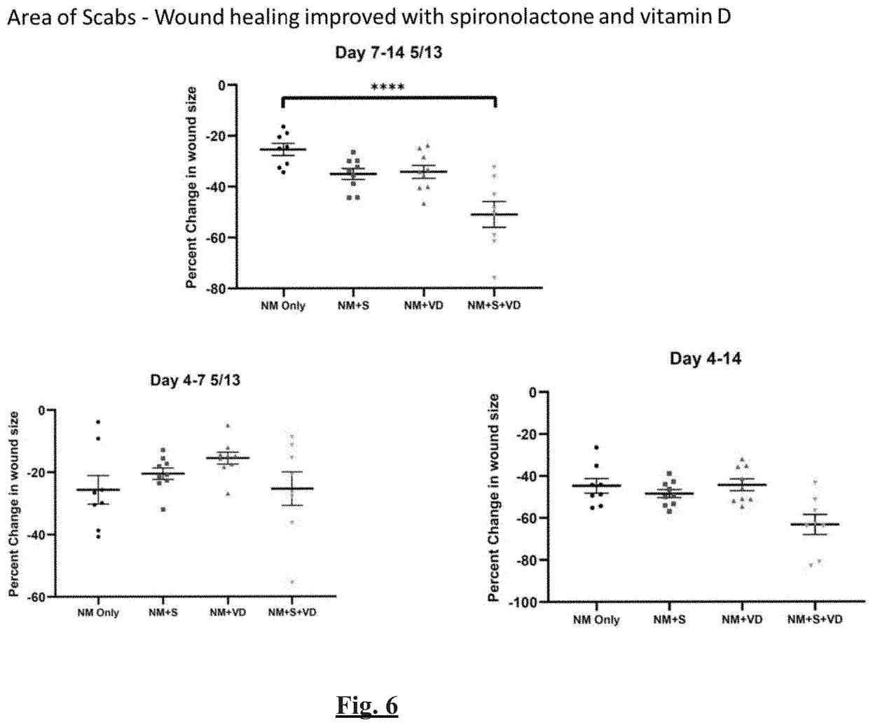

FIG. 6 shows that by day 14, the combination of spironolactone and vitamin D results in 60% reduction in scab/wound area. The combination treatment is better than spironolactone or vitamin D treatment alone.

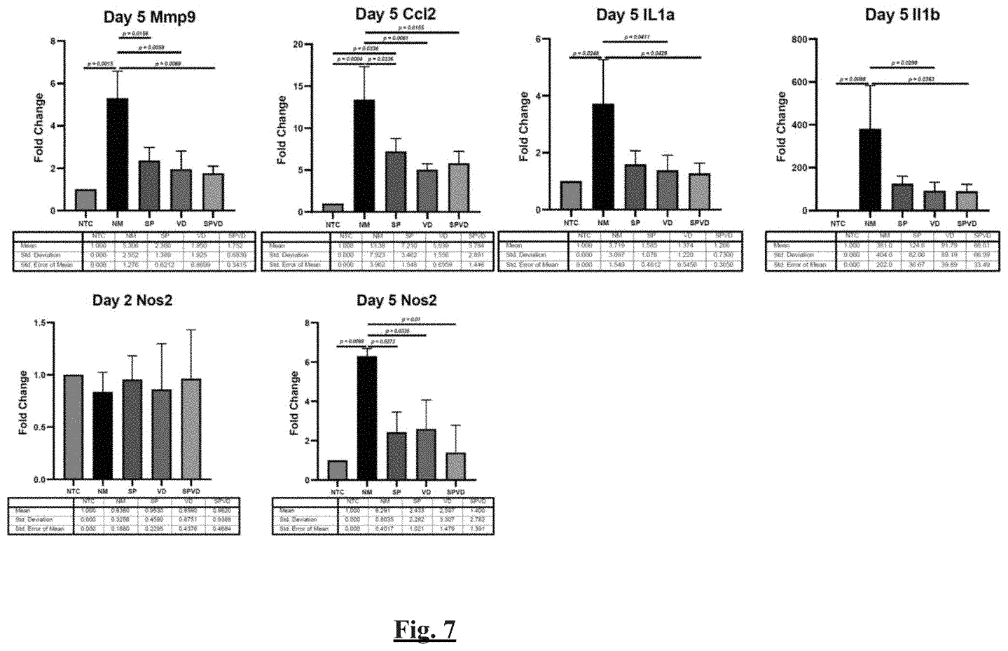

As early as 48 hours we observe a significant 50% reduction in the inflammatory chemokines CCL2 (p=0.03, n=5) (FIG. 7). This hallmark chemokine plays a critical role in recruiting immune cells to damaged skin, specifically macrophages. By day 5, we observe greater than 50-60% reduction in inflammatory factors IL-1a, IL-1b, CCL2. Additionally we observe significant decreases in the tissue destructive factor MMP9 as well as iNOS (66% and 62%, respectively), which are further reduced with combination treatment (26% and 43%, respectively). The combination treatment is significantly different from no treatment control and is trending towards significance compared to spironolactone alone (n=5).

From the above description, those skilled in the art will perceive improvements, changes and modifications. Such improvements, changes, and modifications are within the skill of those in the art and are intended to be covered by the appended claims. All patents, patent applications, and publication cited herein are incorporated by reference in their entirety.

The following is claimed:

1. A composition for preventing or treating skin damage in skin of a mammalian subject in need thereof, the composition comprising:
at least one autophagy promoter; and
at least one diuretic in combination with the at least one autophagy promoter;
wherein the at least one autophagy promoter and the at least one diuretic are each present in amount sufficient to prevent or treat the skin damage.

2. The composition of claim 1, wherein the at least one autophagy promoter is present in an amount sufficient to minimize injury and/or promote healing at the site of skin damage in the subject.

3. The composition of claim 1, wherein the at least one diuretic is present in an amount sufficient to reduce or prevent swelling at the site of the skin damage.

4. The composition of claim 1, wherein the at least one autophagy promoter, in combination with the at least one diuretic, are each present in an amount sufficient to minimize injury and/or promote healing and/or reduce or prevent swelling at the site of the skin damage.

5. The composition of claim 1, being formulated for one of oral, inhalation, sublingual, injection, or topical administration to the subject.

6. The composition of claim 1, wherein the subject is a human subject.

7. The composition of claim 1, wherein the skin damage is caused by one or more of a chemical burn (including but not limited to as caused by vesicants), a radiation burn (including but not limited as caused by UV and X-rays), a heat burn (including but not limited as caused by fire, steam, hot objects, and hot liquids), a cold burn, an electrical burn, a friction burn, or trauma.

8. The composition of claim 7, wherein the vesicant is one of a weaponized vesicant (including but not limited mustard gas, nitrogen mustard, and sulfur mustard) or a chemotherapeutic vesicant (including but not limited to mechlorethamine and doxorubicin).

9. The composition of claim 1, wherein the at least one autophagy promoter is an mTOR pathway inhibitor.

10. The composition of claim 9, wherein the at least one autophagy promoter is one of Vitamin D, a Vitamin D analogue, a pharmaceutically active source of Vitamin D, or rapamycin.

11. The composition of claim 1, wherein the at least one autophagy promoter is an mTOR pathway independent autophagy promoter.

12. The composition of claim 11, wherein the at least one autophagy promoter is trehalose.

13. The composition of claim 1, wherein the at least one diuretic is selected from the group consisting of high ceiling/loop diuretics, thiazides, carbonic anhydrase inhibitors, potassium-sparing diuretics, calcium-sparing diuretics, osmotic diuretics, low ceiling diuretics, xanthine diuretics, and diuretics of other mechanisms.

14. The composition of claim 13, wherein the at least one diuretic is selected from the group consisting of spironolactone, furosemide, hydrochlorothiazide, pamabrom, caffeine, and stevia.

15. A method for preventing or treating skin damage in skin of a mammalian subject in need thereof, the method comprising administering to the subject a therapeutically effective amount of the composition of claim 1, thereby preventing or treating the skin damage in the subject.

16. The method of claim 15, wherein the administration step includes oral, inhalation, sublingual, injection, or topical administration of the composition to the subject.

17. The method of claim 15, wherein the subject is a human subject.

18. The method of claim 15, wherein the skin damage is caused by one or more of a chemical burn (including but not limited to as caused by vesicants), a radiation burn (including but not limited as caused by UV and X-rays), a heat burn (including but not limited as caused by fire, steam, hot objects, and hot liquids), a cold burn, an electrical burn, a friction burn, or trauma.

19. (canceled)

20. (canceled)

21. (canceled)

22. (canceled)

23. (canceled)

24. (canceled)

25. (canceled)

26. A method for preventing or treating skin damage in skin of a mammalian subject in need thereof, the method comprising:
(i) administering, to the subject, a therapeutically effective amount of the composition of claim 1; and
(ii) administering, after step (i), a therapeutically effect amount of a series of diuretic compositions on separate consecutive days to the subject;
whereby steps (i) and (ii) are effective to prevent or treat the skin damage in the subject.

27. (canceled)

28. (canceled)

29. (canceled)

30. (canceled)

31. (canceled)

32. A kit for preventing or treating skin damage in skin of a mammalian subject in need thereof, the kit comprising:
the composition of claim 1, being formulated for oral administration;
a series of diuretic compositions, each of which is formulated for oral administration; and
a set of dosing instructions, wherein the subject is instructed to (i) ingest the composition of claim 1 on a first day, and (ii) subsequently ingest each of the diuretic compositions on separate consecutive days.

33. (canceled)

34. (canceled)

35. (canceled)

36. (canceled)

37. (canceled)
